(19) United States
(12) Patent Application Publication
(10) Pub. No.: US 2023/0200775 A1
(43) Pub. Date: Jun. 29, 2023

(54) ULTRASONIC IMAGING SYSTEM

(71) Applicant: NaviFUS Co., Ltd., Taipei City (TW)

(72) Inventors: Hao-Li LIU, Taoyuan City (TW); Po-Hsiang TSUI, Taoyuan City (TW); Chi-Chao LEE, Taoyuan City (TW)

(21) Appl. No.: 18/117,437

(22) Filed: Mar. 4, 2023

Related U.S. Application Data
(62) Division of application No. 16/864,530, filed on May 1, 2020.

(30) Foreign Application Priority Data
Sep. 10, 2019 (TW) 108132547

(51) Int. Cl. A61B 8/14; A61B 6/00; A61B 5/00; A61B 5/055; A61B 8/08; A61B 6/03

(52) U.S. Cl. CPC A61B 8/14; A61B 6/5247; A61B 5/0035; A61B 5/055; A61B 8/483; A61B 8/5261; A61B 6/032; A61B 2562/08

(57) ABSTRACT

An ultrasonic imaging system includes an ultrasonic probe and a processing unit. The ultrasonic probe is operable at multiple different tilt angles to perform ultrasonic measurement and to obtain a plurality 2D ultrasonic images corresponding respectively to the different tilt angles. The processing unit calculates a 3D ultrasonic images based on the 2D ultrasonic images and the corresponding tilt angles.

### CROSS-REFERENCE TO RELATED APPLICATION

This application is a divisional patent application of U.S. patent application Ser. No. 16/864,530, which claims priority to Taiwanese Invention Patent Application No. 108132547, filed on Sep. 10, 2019.

### FIELD

The disclosure relates to an imaging system, and more particularly to an ultrasonic imaging system.

### BACKGROUND

Ultrasound imaging is now widely used clinically for tissue diagnosis. A crystal of a conventional ultrasonic diagnostic probe can achieve a one-dimensional (1D) array arrangement with linear cutting, so directional electronic phase focusing can be performed to create a two-dimensional (2D) sectional image (ultrasonic image).

Since the conventional ultrasound imaging can only generate 2D ultrasonic images, one conventional approach to obtaining a three-dimensional (3D) ultrasonic image moves an ultrasonic probe to perform manual scanning so as to acquire multiple sectional images corresponding to different locations in sequence, and then performs numerical operations on the acquired sectional images to construct the 3D ultrasonic image. An array ultrasonic probe with 2D cutting may also be used to acquire the sectional images corresponding to different locations by having the ultrasonic probe elements be excited row by row. However, the probe used in the first approach may be expensive because of the high complexity in mechanical design, and the probe used in the second approach may be even more expensive.

### SUMMARY

Obtaining 3D anatomical information is critical for clinical interventional judgment. In this disclosure, it is intended to propose two possible approaches to providing 3D anatomical information for image guided intervention. The first one is to obtain 3D anatomical information via real-time reconstruction of 3D ultrasonic images. The second one is to superimpose a 2D real-time ultrasonic image onto a high-resolution 3D medical image.

Therefore, an object of the disclosure is to provide an ultrasonic imaging system that is used to construct a 3D ultrasonic image.

According to the disclosure, the ultrasonic imaging system includes an ultrasonic probe and a processing unit electrically coupled to the ultrasonic probe. The ultrasonic probe is operable at multiple different tilt angles that are defined by coplanar lines to send ultrasonic signals into a test target and to receive reflected ultrasonic signals corresponding to the ultrasonic signals from the test target. The processing unit controls the ultrasonic probe to send the ultrasonic signals and to receive the reflected ultrasonic signals, and is configured to generate a plurality of 2D ultrasonic images that respectively correspond to the different tilt angles based on the reflected ultrasonic signals, and to generate a 3D ultrasonic image based on the 2D ultrasonic images and the different tilt angles.

Another object of the disclosure is to provide an ultrasonic imaging system that can construct a 3D ultrasonic image and superimpose the constructed 3D ultrasonic image with a 3D medical image.

According to the disclosure, the ultrasonic imaging system includes an ultrasonic probe, a processing unit electrically coupled to the ultrasonic probe, a first pattern fixed on the ultrasonic probe, a second pattern to be disposed on the test target, a storage unit electrically coupled to the processing unit, an image capturing unit electrically coupled to the processing unit, and a display unit electrically coupled to the processing unit. The ultrasonic probe is operable at multiple different tilt angles that are defined by coplanar lines to send ultrasonic signals into a test target and to receive reflected ultrasonic signals corresponding to the ultrasonic signals from the test target. The processing unit controls the ultrasonic probe to send the ultrasonic signals and to receive the reflected ultrasonic signals, and is configured to generate a plurality of 2D ultrasonic images that respectively correspond to the different tilt angles based on the reflected ultrasonic signals, and to generate a 3D ultrasonic image based on the 2D ultrasonic images and the different tilt angles. The second pattern has a predefined fixed positional relationship with the test target. The storage unit stores a 3D image related to the test target, a first positional relationship between the first pattern and each of the 2D ultrasonic images, and a second positional relationship between the second pattern and the test target. The image capturing unit is disposed to capture images of the test target, the first pattern and the second pattern in a real time manner. The processing unit is further configured to obtain a first spatial position-orientation of the first pattern based on the first pattern in the images captured by the image capturing unit, and to acquire a spatial location of the 3D ultrasonic image based on the first positional relationship and the first spatial position-orientation. The processing unit is further configured to obtain a second spatial position-orientation of the second pattern based on the second pattern in the images captured by the image capturing unit, and to acquire a spatial location of the test target based on the second positional relationship and the second spatial position-orientation. The processing unit is further configured to superimpose the 3D ultrasonic image and the 3D image stored in the storage unit together based on the spatial location of the 3D ultrasonic image and the spatial location of the test target.

Yet another object of the disclosure is to provide an ultrasonic imaging system that can superimpose a 2D ultrasonic image with a 3D medical image.

According to the disclosure, the ultrasonic imaging system includes an ultrasonic probe, a processing unit electrically coupled to the ultrasonic probe, a first pattern fixed on the ultrasonic probe, a second pattern to be disposed on the test target, a storage unit electrically coupled to the processing unit, an image capturing unit electrically coupled to the processing unit, and a display unit electrically coupled to the processing unit. The ultrasonic probe is operable to send ultrasonic signals into a test target and to receive reflected ultrasonic signals corresponding to the ultrasonic signals from the test target. The processing unit controls the ultrasonic probe to send the ultrasonic signals and to receive the reflected ultrasonic signals, and is configured to generate a 2D ultrasonic image based on the reflected ultrasonic signals. The second pattern has a predefined fixed positional relationship with the test target. The storage unit stores a 3D image related to the test target, a first positional relationship between the first pattern and the 2D ultrasonic image, and a second positional relationship between the second pattern and the test target. The image capturing unit is disposed to capture images of the test target, the first pattern and the second pattern in a real time manner. The processing unit is further configured to obtain a first spatial position-orientation of the first pattern based on the first pattern in the images captured by the image capturing unit, and to acquire a spatial location of the 2D ultrasonic image based on the first positional relationship and the first spatial position-orientation. The processing unit is further configured to obtain a second spatial position-orientation of the second pattern based on the second pattern in the images captured by the image capturing unit, and to acquire a spatial location of the test target based on the second positional relationship and the second spatial position-orientation. The processing unit is further configured to superimpose the 2D ultrasonic image and the 3D image stored in the storage unit together based on the spatial location of the 2D ultrasonic image and the spatial location of the test target.

### BRIEF DESCRIPTION OF THE DRAWINGS

Other features and advantages of the disclosure will become apparent in the following detailed description of the embodiment(s) with reference to the accompanying drawings, of which:

FIG. 1 is a schematic diagram illustrating a first embodiment of an ultrasonic imaging system according to the disclosure;

FIG. 2 is a perspective view that shows how 2D ultrasonic images are arranged in position to form a 3D ultrasonic image according to this disclosure;

FIG. 3 is a schematic diagram illustrating a front view of FIG. 2;

FIG. 4 is a schematic diagram exemplarily illustrating a relationship between an image plane and a corresponding 2D ultrasonic image;

FIG. 5 is a schematic diagram illustrating a second embodiment of an ultrasonic imaging system according to the disclosure; and

FIG. 6 is a schematic diagram exemplarily illustrating a superimposition of a 2D ultrasonic image and a 3D medical image.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

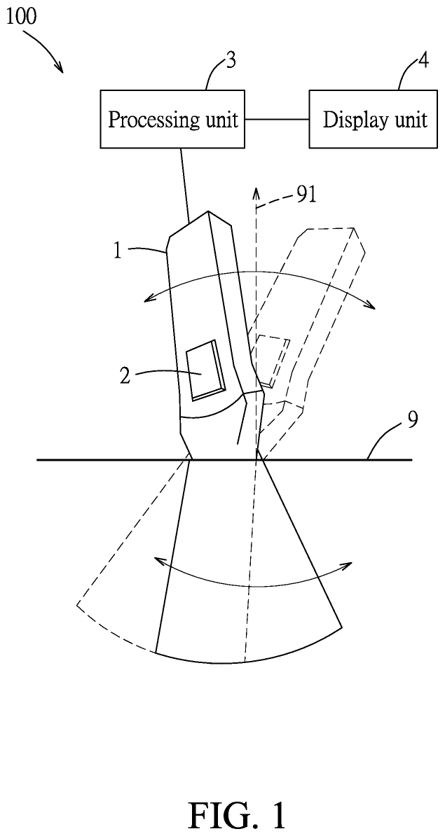

Referring to FIG. 1, a first embodiment of an ultrasonic imaging system 100 according to this disclosure is adapted for use on a test surface 9 of a test target (e.g., a skin surface of a person or an animal, etc., but this disclosure is not limited in this respect), and includes an ultrasonic probe 1, an inertial measurement unit (IMU) 2, a processing unit 3 and a display unit. A reference numeral 91 is used to denote a normal vector of the test surface 9.

The ultrasonic probe 1 may be a conventional ultrasonic probe, and is operable at multiple different tilt angles that are defined by coplanar lines to send ultrasonic signals into the test target and to receive reflected ultrasonic signals corresponding to the ultrasonic signals from the test target. It should be noted that the ultrasonic probe 1 may be held in a user's hand to operate at different tilt angles in some embodiments, or may be operated using a special mechanical device to change among the different tilt angles more steadily in other embodiments.

The IMU 2 is mounted to the ultrasonic probe 1 in such a way that the IMU 2 tilts at a same angle as the ultrasonic probe 1, and is configured to detect acceleration components respectively corresponding to three axial directions that are defined with respect to the IMU 2. In this embodiment, the acceleration components include a first acceleration component, a second acceleration component, and a third acceleration component that respectively correspond to a first axial direction, a second axial direction, and a third axial direction that are perpendicular to each other. The tilt angle is defined to be an angle between the third axial direction and a direction of the gravitational acceleration, and can be anywhere between −90° and 90°. In this embodiment, when the tilt angle is 0°, the third axial direction is parallel to the normal vector 91, but this disclosure is not limited in this respect. The tilt angle, the gravitational acceleration, and the acceleration components have the following relationships:

$$G = \sqrt{{A}_{1}^{2} + {A}_{2}^{2} + {A}_{3}^{2}} \qquad ( 1 )$$

$$φ = {cos}^{- 1} ( \frac{{A}_{3}}{G} ) = {sin}^{- 1} ( \frac{\sqrt{{A}_{1}^{2} + {A}_{2}^{2}}}{G} ) \qquad ( 2 )$$

where G represents a magnitude of the gravitational acceleration, A₁ represents a magnitude of the first acceleration component, A₂ represents a magnitude of the second acceleration component, A₃ represents a magnitude of the third acceleration component, and φ represents the tilt angle. In other words, the tilt angle of the ultrasonic probe 1 can be calculated using the equations (1) and (2).

The processing unit 3 may be a processor of a computer, a digital signal processor (DSP), or any other kind of processing chip having computational capability, but this disclosure is not limited in this respect. The processing unit 3 is electrically coupled to the ultrasonic probe 1 and the IMU 2. When the ultrasonic probe 1 is in operation, the processing unit 3 receives the acceleration components detected by the IMU 2, controls the ultrasonic probe 1 to send the ultrasonic signals and to receive the reflected ultrasonic signals, and then generates a 2D ultrasonic image based on the reflected ultrasonic signals thus received. The 2D ultrasonic image may be a brightness mode (B-Mode) image that is obtainable using a conventional ultrasonic probe, and corresponds to a tilt angle the ultrasonic probe 1 was at when the 2D ultrasonic image was generated. Therefore, the processing unit 3 would generate a plurality of 2D ultrasonic images respectively corresponding to multiple different tilt angles based on the reflected ultrasonic signals received thereby when the ultrasonic probe 1 changes among these different tilt angles during operation. Subsequently, the processing unit 3 calculates, for each of the 2D ultrasonic images, the corresponding tilt angle based on the acceleration components received when the ultrasonic probe 1 was at the corresponding tilt angle (or when the 2D ultrasonic image was generated), and generates a 3D ultrasonic image based on the 2D ultrasonic images and the corresponding tilt angles thus calculated. It is noted that, in some embodiments, it may be the IMU 2 that calculates the tilt angle, and this disclosure is not limited in this respect.

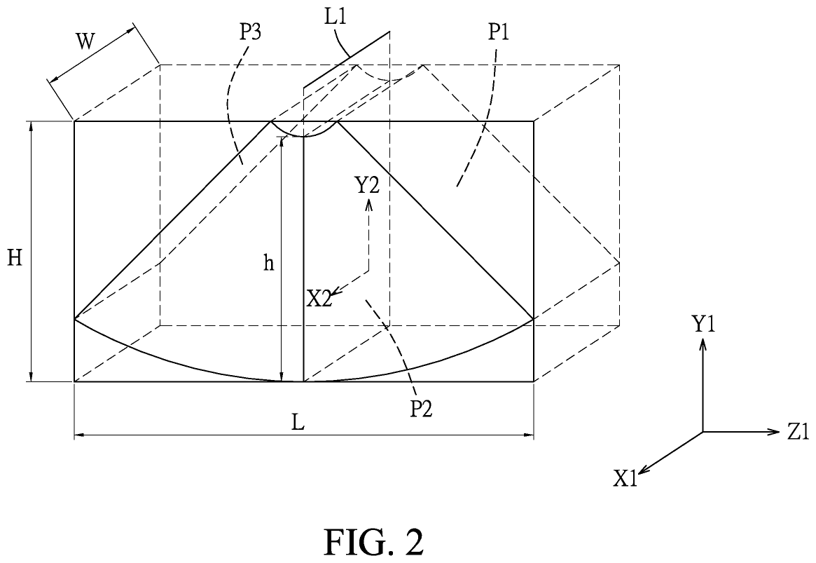

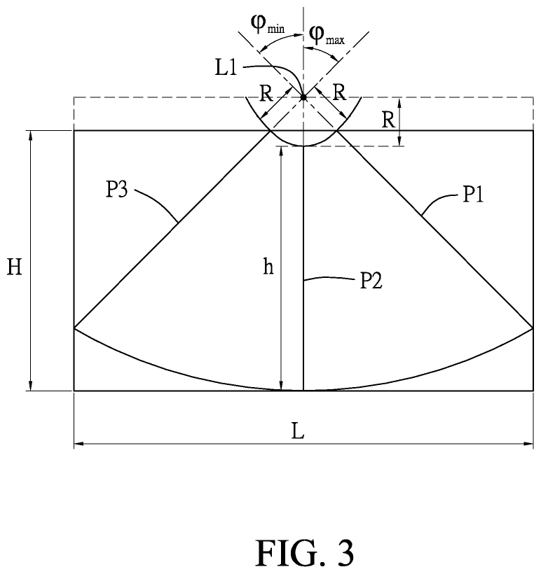

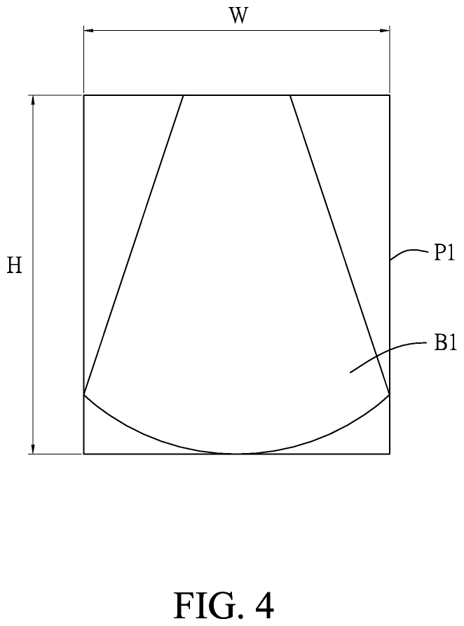

Referring to FIGS. 1 to 3, where FIG. 2 is a perspective view that shows how the 2D ultrasonic images, which respectively correspond to multiple sections of the test target and respectively correspond to multiple image planes, are arranged in position to form the 3D ultrasonic image, and FIG. 3 is a front view of FIG. 2. For the sake of explanation, FIGS. 2 and 3 exemplarily show three image planes (P1, P2, P3) respectively of three 2D ultrasonic images that respectively correspond to the greatest positive tilt angle φₘₐₓ, a tilt angle of 0°, and the greatest negative tilt angle φₘᵢₙ, but in practice, more than three 2D ultrasonic images of which the corresponding tilt angles are between φₘᵢₙ and φₘₐₓ may be generated using the ultrasonic probe 1 and the processing unit 3 in order to form a single 3D ultrasonic image. FIG. 4 exemplarily illustrates a relationship between the image plane (P1) and the corresponding 2D ultrasonic image (B1).

Referring to FIGS. 1 and 2, the image planes corresponding to the 2D ultrasonic images are perpendicular to a plane corresponding to the tilt angles (i.e., a swinging plane of the ultrasonic probe 1), and join on a straight line (L1) on which a crystal (namely, a transmitter for transmitting the ultrasonic signals) of the ultrasonic probe 1 was located during the ultrasonic detection at the multiple tilt angles. The straight line (L1) is spaced apart from each of the 2D ultrasonic images by a fixed distance denoted by R in FIG. 3, where R≥0. In a case that the crystal of the ultrasonic probe 1 is located substantially at a contact surface of the ultrasonic probe 1 that touches the test surface 9 during operation, R=0.

For ease of calculation, in this embodiment, the greatest positive tilt angle φₘₐₓ and the greatest negative tilt angle φₘᵢₙ may have the same magnitude but with different signs. For example, in a case that the greatest positive tilt angle is 60 degrees, the greatest negative tilt angle would be −60 degrees, but this disclosure is not limited thereto. In other cases, the greatest positive tilt angle can be about 90 degrees, and the greatest negative tilt angle would be about −90 degrees.

A maximum width (denoted as W in FIGS. 2 and 4) of the 3D ultrasonic image is equal to a maximum width of each of the 2D ultrasonic images, and the 2D ultrasonic images and the 3D ultrasonic image have dimensional relationships of:

H=h+R(1−sin(φ_{cri})) (3)

L=2(h+R)|cos(φ_{cri})| (4)

where h represents a maximum height of each of the 2D ultrasonic images, H represents a maximum height of the 3D ultrasonic image, L represents a maximum length of the 3D ultrasonic image, and φ_{cri} represents an absolute value of the greatest (greatest when looking at the magnitude only) one of the tilt angles that respectively correspond to the 2D ultrasonic images.

Each of the 2D ultrasonic images corresponds to a respective 2D coordinate system which is defined by an x-axis and a y-axis, and in which the maximum width of the 2D ultrasonic image refers to the maximum width of the 2D ultrasonic image in a direction of the x-axis, and the maximum height of the 2D ultrasonic image refers to the maximum height of the 2D ultrasonic image in a direction of the y-axis. The 3D ultrasonic image corresponds to a 3D coordinate system which is defined by an X-axis, a Y-axis and a Z-axis. As exemplified in FIG. 2, for the 2D ultrasonic image that corresponds to the image plane (P2), the x-axis direction and the y-axis direction of the respective 2D coordinate system are denoted as X2 and Y2, respectively, and the X-axis direction, Y-axis direction and the Z-axis direction of the 3D coordinate system are denoted as X1, Y1 and Z1, respectively. For each of the 2D ultrasonic images, coordinates (x, y) in the respective 2D coordinate system and coordinates (X, Y, Z) in the 3D coordinate system have a relationship defined by:

$$X = x \qquad ( 5 )$$

$$y = ( R + y ) * sin ( φ ) - R * sin ( {φ}_{cri} ) \qquad ( 6 )$$

$$Z = \frac{L}{2} + ( R + y ) * cos ( φ ) \qquad ( 7 )$$

In other embodiments, the ultrasonic imaging system may acquire the tilt angle of the ultrasonic probe in ways other than using the IMU. For example, the ultrasonic imaging system may include a camera, and the ultrasonic probe may be provided with a barcode or a specific pattern thereon. Image recognition techniques may be used on an image captured by the camera of the barcode or the specific pattern in order to obtain Euler angles of the ultrasonic probe, and then acquire a corresponding tilt angle accordingly. In another example, the ultrasonic imaging system may include two cameras, and use an angular difference between the cameras to construct a location of the ultrasonic probe in the 3D space, thereby obtaining the Euler angles and the tilt angle of the ultrasonic probe. In yet another example, the ultrasonic imaging system may include an electromagnetic tracker that uses magnetic induction to identify three dimensional directions, so as to obtain the Euler angles and the tilt angle of the ultrasonic probe.

The display unit 4 is exemplified as a screen that is electrically coupled to the processing unit 3 for displaying the 3D ultrasonic image, or for displaying the 3D ultrasonic image and the 2D ultrasonic images simultaneously. In some embodiments, the processing unit 3 may be capable of generating a sectional image by taking a sectional view of the 3D ultrasonic image in any desired direction, of performing image processing on the sectional image, and of causing the display unit 4 to display the sectional image and the result of the image processing at the same time.

The processing unit 3 may perform image processing on the sectional image to generate functional images of, for example, entropy-based imaging, Doppler imaging, strain imaging, Nakagami imaging, and so on. The functional images of Doppler imaging may show blood flow. The functional images of strain imaging may be provided for Young's modulus measurement to identify elasticity of tissue. The functional images of entropy-based imaging or Nakagami imaging may provide analysis of regularity in structural arrangement of tissue. The processing unit 3 can cause the display unit 4 to simultaneously display the sectional image and at least one of the functional images, the 3D ultrasonic image and the 2D ultrasonic images, thereby providing various different ultrasound-based images for inspection by medical professionals.

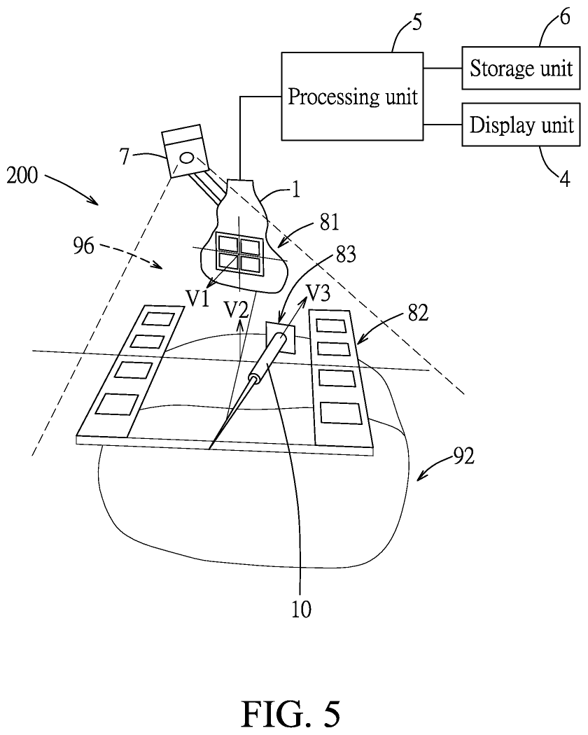

Referring to FIG. 5, a second embodiment of an ultrasonic imaging system 200 according to this disclosure is adapted for use on a test surface of a test target 92, and includes an ultrasonic probe 1, an intervention tool 10 (e.g., a puncture needle, a syringe, a surgical knife, etc.), a first pattern 81, a second pattern 82, a third pattern 83, a display unit 4, a processing unit 5, a storage unit 6, and an image capturing unit 7. In this embodiment, the test target 92 is exemplified as an abdomen of a human body. The ultrasonic probe 1 is operated to generate the ultrasonic signals and to receive the reflected ultrasonic signals for the processing unit 5 to generate 2D ultrasonic images. The ultrasonic imaging system 200 may superimpose a 2D ultrasonic image or a constructed 3D ultrasonic image onto other kinds of structural medical images, such as images of magnetic resonance imaging (MRI), computerized tomography (CT), etc., for assisting clinicians in diagnosis.

The first pattern 81 is fixed on the ultrasonic probe 1.

The second pattern 82 is disposed on the test target 92 in such a way that the second pattern 82 has a predefined fixed positional relationship with the test target 92.

The third pattern 83 is disposed on the intervention tool 10.

Each of the first pattern 81, the second pattern 82 and the third pattern 83 includes one or more one-dimensional barcodes, or one or more two-dimensional barcodes, or a specific pattern that is adapted for acquiring, via image recognition, a fixed normal vector (i.e., a normal vector with a fixed initial point, representing a spatial position and a spatial orientation) of the specific pattern. The fixed normal vector may include information of spatial position, orientation, and angle of the fixed normal vector in the 3D space.

In this embodiment, the first pattern 81 is exemplified to include four square two-dimensional barcodes, the second pattern 82 is exemplified to include eight coplanar two-dimensional barcodes that are disposed at two opposite sides of the test surface of the test target 92, and the third pattern 83 is exemplified to include one two-dimensional barcode that is attached to the intervention tool 10. However, in FIG. 5, the first pattern 81, the second pattern 82, and the third pattern 83 are simply illustrated as four blank squares, eight blank squares, and one blank square for the sake of simplicity of illustration, although they in fact have predesigned two-dimensional barcodes therein.

The storage unit 6 is electrically coupled to the processing unit 5, and stores a 3D image related to the test target 92, a first positional relationship between the first pattern 81 and each of the 2D ultrasonic images, a second positional relationship between the second pattern 82 and the test target 92, and a third positional relationship between the third pattern 83 and the intervention tool 10. The 3D image has a high resolution, and may be a medical image of, for example, computerized tomography (CT), magnetic resonance imaging (MRI), etc. The first positional relationship between the first pattern 81 and each of the 2D ultrasonic images is fixed because the first pattern 81 is fixed on the ultrasonic probe 1 and moves along with the ultrasonic probe 1. The second positional relationship between the second pattern 82 and the test target 92 is fixed since the second pattern 82 is positioned on the test target 92 in a predefined manner. The third positional relationship between the third pattern 83 and the intervention tool 10 is fixed since the third pattern 83 is positioned on the intervention tool 10. Accordingly, the first positional relationship, the second positional relationship and the third positional relationship are predesigned or known parameters in this embodiment.

The image capturing unit 7 (e.g., a digital camera) is electrically coupled to the processing unit 5, and is disposed to capture images of the test target 92, the first pattern 81, the second pattern 82 and the third pattern 83 in a real time manner. That is, the test target 92, the first pattern 81, the second pattern 82 and the third pattern 83 are all covered by a field of view of the image capturing unit 7. In this embodiment, the image capturing unit 7 is mounted to the ultrasonic probe 1, but this is not essential for this embodiment as long as the image captured by the image capturing unit 7 can include the test target 92, the first pattern 81 and the second pattern 82 at the same time. For example, the image capturing unit 7 can be mounted to the test target 92 or the intervention tool 10 in other embodiments. A number of lenses of the image capturing unit 7 is determined using an image recognition and analysis technique to ensure that identification of a position and an orientation of the first pattern 81 (referred to as first spatial position-orientation hereinafter, and denoted as a fixed normal vector (V1) of a plane corresponding to the first pattern 81 in FIG. 5), a position and an orientation of the second pattern 82 (referred to as second spatial position-orientation hereinafter, and denoted as a fixed normal vector (V2) of a plane corresponding to the second pattern 82 in FIG. 5), and a position and an orientation of the third pattern 83 (referred to as third spatial position-orientation hereinafter, and denoted as a fixed normal vector (V3) of a plane corresponding to the third pattern 83 in FIG. 5) can be performed. The first spatial position-orientation (V1), the second spatial position-orientation (V2) and the third spatial position-orientation (V3) may be defined with reference to the image capturing unit 7 or a preset reference point.

The processing unit 5 obtains the first spatial position-orientation (V1) of the first pattern 81 based on the first pattern 81 in images captured by the image capturing unit 7, obtains the second spatial position-orientation (V2) of the second pattern 82 based on the second pattern 82 in the images captured by the image capturing unit 7, and obtains the third spatial position-orientation (V3) of the third pattern 83 based on the third pattern 83 in the images captured by the image capturing unit 7. In one embodiment, the processing unit 5 is a part of the image capturing unit 7.

In more detail, each of the images captured by the image capturing unit 7 contains all of the two-dimensional barcodes of the plurality of patterns 81, 82, 83, and each two-dimensional barcode may include at least three identification points that are disposed at specific positions (e.g., edges, corners, the center, etc.) of the two-dimensional barcode, respectively. When the processing unit 5 successfully identifies the identification points, the processing unit 5 uses predetermined or known spatial/positional relationships among the image capturing unit 7 and the identification points to acquire positional information of each of the identification points in the 3D space, and assigns spatial coordinates to each of the identification points accordingly.

Subsequently, for each of the two-dimensional barcodes, the processing unit 5 calculates a spatial vector for any two of the identification points of the two-dimensional barcode. The at least three identification points of the two-dimensional barcode may correspond to at least two distinct spatial vectors that are coplanar with the two-dimensional barcode. The processing unit 5 then calculates a cross product of two of the at least two spatial vectors for the two-dimensional barcode, thereby acquiring a fixed normal vector for the two-dimensional barcode. In another embodiment, the processing unit 5 calculates cross products for any two of the spatial vectors for the two-dimensional barcode, and acquires an average of the cross products to obtain a representative fixed normal vector for the two-dimensional barcode. In one implementation, one of the identification points of the two-dimensional barcode may be disposed at the center of the two-dimensional barcode, so the fixed normal vector calculated based on two spatial vectors corresponding to the central one of the identification points would be located at the center of the two-dimensional barcode. In other cases, if each two-dimensional barcode is below a certain size (sufficiently small) and has at least a certain number of identification points (sufficient number of identification points), the representative fixed normal vector of the two-dimensional barcode acquired based on the average of the cross products would be close to the center of the two-dimensional barcode. The processing unit 5 calculates an average of the fixed normal vectors (or the representative fixed normal vectors) obtained for the two-dimensional barcodes of the first pattern 81 to obtain the first spatial position-orientation (V1) of the first pattern 81, calculates an average of the fixed normal vectors (or the representative fixed normal vectors) obtained for the two-dimensional barcodes of the second pattern 82 to obtain the second spatial position-orientation (V2) of the second pattern 82, and calculates an average of the fixed normal vectors (or the representative fixed normal vectors) obtained for the two-dimensional barcodes of the third pattern 83 to obtain the third spatial position-orientation (V3) of the third pattern 83.

In this embodiment, since the second spatial position-orientation (V2) is obtained based on the eight two-dimensional barcodes, each of which has a set of known spatial coordinates, the processing unit 5 can acquire representative spatial coordinates of the first spatial position-orientation (V1) (e.g., coordinates of an initial point of the fixed normal vector (V1) in FIG. 5), representative spatial coordinates of the second spatial position-orientation (V2) (e.g., coordinates of an initial point of the fixed normal vector (V2) in FIG. 5), and representative spatial coordinates of the third spatial position-orientation (V3) (e.g., coordinates of an initial point of the fixed normal vector (V3) in FIG. 5) via, for example, a transformation matrix and/or a scale factor.

After acquiring the first spatial position-orientation (V1) based on the first pattern 81 in the images captured by the image capturing unit 7, the processing unit 5 acquires a spatial location of a corresponding 2D ultrasonic image based on the first positional relationship and the first spatial position-orientation (V1). After acquiring the second spatial position-orientation (V2) based on the second pattern 82 in the images captured by the image capturing unit 7, the processing unit 5 acquires a spatial location of the test target 92 based on the second positional relationship and the second spatial position-orientation (V2). After acquiring the third spatial position-orientation (V3) based on the third pattern 83 in the images captured by the image capturing unit 7, the processing unit 5 acquires a spatial location of the intervention tool 10 based on the third positional relationship and the third spatial position-orientation (V3). Subsequently, the processing unit 5 superimposes the 2D ultrasonic image and the 3D image stored in the storage unit 6 together based on the spatial location of the 2D ultrasonic image and the spatial location of the test target 92, superimposes an image of the intervention tool 10 on the 3D image based on the spatial location of the intervention tool 10 and the spatial location of the test target 92, and causes the display unit 4 that is electrically coupled to the processing unit 5 to display the resultant image.

It is noted that, in some embodiments where the image of the intervention tool 10 is not required to be shown in the resultant image, the third pattern 83 may be omitted.

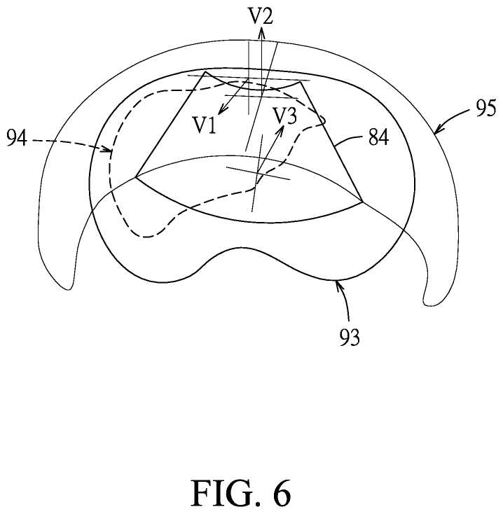

FIG. 6 exemplarily illustrates the superimposition of the 2D ultrasonic image 84 and the 3D image (i.e., the resultant image) that contains images of a rib portion 93, a liver portion 94 and a skin portion 95 of the test target 92 (i.e., the abdomen in this embodiment). The 3D image may be constructed by performing image analysis and feature extraction on multiple images of CT or MRI. In this embodiment, the rib portion 93, the liver portion 94 and the skin portion 95 and their 3D profiles are the features extracted from the images of CT or MRI. One typical embodiment is that a procedure is added to align the 3-D CT/MRI image with the real body anatomy (abdomen in this example) by manually redefining the reference coordinates of V1, V2, V3, . . . to new positions so that the 3D image-reconstructed organ can perfectly align with the real organ of the patient.

Furthermore, in some implementations of the second embodiment, the ultrasonic imaging system 200 may generate a 3D ultrasonic image using the method introduced in the first embodiment, and the processing unit 5 may superimpose the 3D ultrasonic image and the 3D image stored in the storage unit 6 together based on a spatial location of the 3D ultrasonic image and the spatial location of the test target 92. It is noted that since the 3D ultrasonic image is generated based on the 2D ultrasonic images obtained at multiple different tilt angles, the spatial location of the 3D ultrasonic image can be acquired based on the first positional relationship.

In summary, the first embodiment according to this disclosure uses the IMU 2 to acquire the tilt angle of the ultrasonic probe 1, so as to generate the 3D ultrasonic image based on the 2D ultrasonic images obtained at different tilt angles. The first embodiment can easily be applied to the conventional mid-end and low-end ultrasonic imaging systems with low cost and low complexity. The second embodiment according to this disclosure uses the image capturing unit 7 and preset patterns 81, 82, 83 to acquire positional relationships among the 3D medical image, the 2D/3D ultrasonic image and the intervention tool 10, so as to superimpose the 3D medical image, the 2D/3D ultrasonic image and the image of the intervention tool 10 together. The resultant image may have both the advantage of the high resolution from the 3D medical image and the advantage of immediacy from the 2D/3D ultrasonic image, thereby facilitating clinical diagnosis and treatment.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. An ultrasonic imaging system, comprising:
an ultrasonic probe operable at multiple different tilt angles that are defined by coplanar lines to send ultrasonic signals into a test target and to receive reflected ultrasonic signals corresponding to the ultrasonic signals from the test target;
a processing unit electrically coupled to said ultrasonic probe for controlling said ultrasonic probe to send the ultrasonic signals and to receive the reflected ultrasonic signals, and configured to generate a plurality of two-dimensional (2D) ultrasonic images that respectively correspond to the different tilt angles based on the reflected ultrasonic signals, and to generate a three-dimensional (3D) ultrasonic image based on the 2D ultrasonic images and the different tilt angles;
a first pattern fixed on said ultrasonic probe;
a second pattern to be disposed on the test target in such a way that said second pattern has a predefined fixed positional relationship with the test target;
a storage unit electrically coupled to said processing unit, and storing a 3D image related to the test target, a first positional relationship between said first pattern and each of the 2D ultrasonic images, and a second positional relationship between said second pattern and the test target;
an image capturing unit electrically coupled to said processing unit, and disposed to capture images of the test target, said first pattern and said second pattern in a real time manner; and
a display unit electrically coupled to said processing unit;
wherein said processing unit is further configured to obtain a first spatial position-orientation of said first pattern based on said first pattern in the images captured by said image capturing unit, and to acquire a spatial location of the 3D ultrasonic image based on the first positional relationship and the first spatial position-orientation;
wherein said processing unit is further configured to obtain a second spatial position-orientation of said second pattern based on said second pattern in the images captured by said image capturing unit, and to acquire a spatial location of the test target based on the second positional relationship and the second spatial position-orientation; and
wherein said processing unit is further configured to superimpose the 3D ultrasonic image and the 3D image stored in said storage unit together based on the spatial location of the 3D ultrasonic image and the spatial location of the test target.

2. The ultrasonic imaging system of claim 1, further comprising an inertial measurement unit (IMU) mounted to said ultrasonic probe in such a way that said IMU tilts at a same angle as said ultrasonic probe, and configured to detect acceleration components respectively corresponding to three axial directions that are defined with respect to said IMU;
wherein said processing unit is electrically coupled to said IMU for receiving, when said ultrasonic probe is at each of the tilt angles, the acceleration components generated by said IMU at the tilt angle, and calculates the tilt angle based on the acceleration components corresponding to the tilt angle.

3. The ultrasonic imaging system of claim 2, wherein the 3D image of the test target is a medical image obtained using computerized tomography (CT) or magnetic resonance imaging (MRI).

4. The ultrasonic imaging system of claim 2, wherein each of said first pattern and said second pattern includes one of a first barcode group, a second barcode group, and a specific pattern, said first barcode group including multiple one-dimensional barcodes, said second barcode group including multiple two-dimensional barcodes, said specific pattern being adapted for acquiring, via image recognition, a spatial position and a spatial orientation of said specific pattern.

5. The ultrasonic imaging system of claim 2, wherein said image capturing unit is mounted to said ultrasonic probe.
